(19) United States
(12) Patent Application Publication
(10) Pub. No.: US 2025/0356960 A1
(43) Pub. Date: Nov. 20, 2025

(54) OBTAINING DOPANT PACKAGE FOR CATALYSIS

(71) Applicant: SHELL USA, INC., Houston, TX (US)

(72) Inventors: Ligang LU, Houston, TX (US); Benjamin COMER, Houston, TX (US); Peipei SHI, Houston, TX (US); Bradley Paul LAMBETH, JR., Houston, TX (US); Gary James WELLS, Houston, TX (US); John Robert LOCKEMEYER, Houston, TX (US); Huihui YANG, Houston, TX (US)

(21) Appl. No.: 19/182,713

(22) Filed: Apr. 18, 2025

Related U.S. Application Data
(60) Provisional application No. 63/649,503, filed on May 20, 2024.

(51) Int. Cl. G16C 20/70; G16C 20/30

(52) U.S. Cl. CPC G16C 20/70; G16C 20/30

(57) ABSTRACT

A system and method for determining a dopant package for catalysis, wherein the dopant package comprises one or more dopants, each dopant having a dopant amount. The method comprises using a generative model to generate a candidate dopant compound and using a predictive machine learning model to predict performance values associated with a plurality of input dopant packages, wherein at least one of the plurality of input dopant packages includes the candidate dopant compound. A dopant package for catalysis is determined by performing a search based on the predicted performance values.

### CROSS REFERENCE TO RELATED APPLICATIONS

This application claims priority from U.S. provisional Application No. 63/649,503 filed 20 May 2024 which is incorporated herein by reference in its entirety.

### TECHNICAL FIELD

The present invention relates using artificial intelligence to determine dopant packages for catalysis.

### BACKGROUND

Catalysts change the rate of a chemical reaction and can speed up a chemical reaction by lowering the energy barrier to the reaction. Dopants are additives in a catalyst formulation that modify the performance of the catalyst, interacting with the catalyst and/or a carrier to improve performance.

### SUMMARY

This summary is provided to present a selection of concepts disclosed herein in a simplified form, which are described in more detail below. This summary is not intended to identify key features or essential features of the claimed subject matter nor is it intended to be used to limit the scope of the claimed subject matter.

Described herein is a computer-implemented method for determining a dopant package for catalysis, wherein the dopant package comprises one or more dopants, each dopant having a dopant amount. The method comprises using a generative model to generate a candidate dopant compound. Using a generative model to generate a candidate dopant compound means that a new candidate dopant compound is generated. The method also comprises using a predictive machine learning model to predict performance values associated with a plurality of input dopant packages, wherein at least one of the plurality of input dopant packages includes the candidate dopant compound, and determining the dopant package for catalysis by performing a search based on the predicted performance values. Using a predictive machine learning model to predict performance values means that accurate predictions of performance values are obtained.

In example scenarios, the machine learning model has been trained using supervised learning and during training the model parameters are adjusted such that the machine learning model accurately predicts one or plural outcome performance values based on an input dopant package. Using a predictive ML model means that performance values can be predicted without laboratory studies to determine performance values. It also allows for a large number of input dopant packages to be tested and means that that any suitable dopant package can be input into the predictive ML model to be tested. The plurality of input dopant packages includes the candidate dopant compound from the generative model and this means that, once a candidate dopant compound is generated by the generative model, a suitable dopant package including the candidate dopant compound can be determined. By combining the generation of a new dopant compound with searching for a dopant package, an improved dopant package for catalysis is generated which may be used to change the rates of chemical reactions.

In some examples, the plurality of input dopant packages are transformed into functional group space before being input into the predictive ML model. Dimensions in functional group space correspond to functional groups. Because functional groups represent the way a dopant molecule behaves chemically, representing a dopant package in functional group space means that dimensionality can be reduced while most information relating to performance values is maintained. Reducing dimensionality before inputting into the predictive ML model makes the method more efficient.

In other examples the plurality of input dopant packages are defined in the functional group space. This means that the distribution of input dopant packages in functional group space can be selected more accurately. The process is therefore made more efficient because computational resources are saved compared to the scenario where many input dopant packages are input into the predictive ML model which are close together in functional group space.

Various use scenarios include performing the search in functional group space. Performing the search in functional group space means that the predicted performance values of input dopant packages in functional group space can be used directly to perform the search. Once determined in functional group space, the determined dopant package is inverse transformed back into dopant space so that the dopant package for catalysis can be prepared for use.

In various examples the search is an interpolative search, which uses interpolation to obtain performance values of a dopant package which was not part of the input dopant package provided to the predictive ML model. Using an interpolative search means that the search is not restricted to the input dopant packages and therefore the search is improved and is more likely to return a dopant package with improved performance values.

The search may be based on a trend in the performance values or a combination of multiple performance values. This means that the search is improved because the search can follow a desired trend. In various scenarios the search is based on multiple trends in multiple performance values in order to improve the overall performance of the dopant package in catalysis.

In some examples, the plurality of input dopant packages are determined based on a trend in known performance values. This means that input dopant packages are selected which are more likely to have improved performance. It also makes the process more efficient because it can reduce the number of input dopant packages provided to the predictive ML model.

Test conditions may also be determined by providing a plurality of test conditions to the predictive machine learning model along with the input dopant packages, wherein the predictive machine learning model predicts the performance values based on the input test conditions. In such scenarios, the search is performed to find a dopant package along with test conditions. Test conditions also affect the performance of the dopant package during catalysis. Therefore by determining test conditions the process of catalysis can be improved.

In some examples the predictive machine learning model is an ensemble tree based learning model or a neural network model. These and other suitable machine learning models provide accurate performance value predictions based on input dopant packages.

The predictive machine learning model in some examples is trained on a dataset of dopant packages and performance values associated with each dopant package. In various examples the training is supervised training. By training the machine learning model in this way, the parameters of the machine learning model are adjusted so that the model outputs accurate predicted performance values based on an input dopant package.

The predicted values from the machine learning model are performance values which may be related to chemical properties associated with catalysis including activity or selectivity. This means that a determined output dopant compound can be found with performance values suitable for improved catalysis.

The predicted performance values from the machine learning model are optionally displayed in a user interface (UI) for example a graphical user interface (GUI). Displaying the predicted values in a UI allows a user to view and visualize the data. For example, the user uses the UI to visualize how performance values change, either in functional group space or in dopant space

In some scenarios, the user provides input via the UI and the user provided input is used to determine the dopant package. Various examples of those scenarios include: the user provides input which determines the plurality of input dopant packages which are input into the predictive machine learning model, the user provides input which determines the performance values on which to base the search, the user provides input as to whether the search is an interpolative search. The method for determining a dopant package may therefore be improved by allowing user input via the UI.

In various examples, the generative model comprises a learned graph grammar for dopant compounds, wherein the learned graph grammar includes production rules for generating dopant compounds. Using a learned graph grammar as a predictive model means that a smaller training set can be used while maintaining high performance compared to other generative models such as generative pretrained transformer (GPT) based generative models or a junction tree variational autoencoder. In further examples the graph grammar is learned using a neural network.

The candidate dopant compound generated by the generative model is optionally validated by inputting the candidate dopant compound into a large language model (LLM). The LLM is for example ChatGPT (trademark) which has been trained on a very large corpus of training data. The LLM provides an indication of the suitability of the candidate dopant compound for catalysis for example by providing information on the chemical properties of the candidate dopant compound, the availability of the candidate dopant compound or how to produce the candidate dopant compound. Validating the candidate dopant compound once it has been generated by the generative model provides a means to check that the dopant compound will be suitable for inclusion in a dopant package for catalysis, before it is included in a plurality of input dopant packages to be input into the predictive ML model.

The methods described herein sometimes include obtaining the generative model using a dopant compound training dataset comprising a plurality of dopant compounds, wherein at least one of the plurality of dopant compounds in the dopant compound training set is determined by using a large language model (LLM) to extract dopant information from one or more dopants. This leads to an increase in the size of the database used to train the generative model which results in improved performance of the model and therefore improved generated candidate dopant molecules.

In some examples the dopant information is extracted using the LLM based on a prompt provided by a user. The prompt is determined such that the LLM produces relevant information from the documents.

In further examples, the dopant information is extracted from a data table in the one or more documents. Data tables provide structured information which is often useful dopant compound data.

Also described herein is a method for selecting one or more dopant compounds for catalysis. The method comprises generating a plurality of dopant compounds using a generative model. Using a generative model to generate dopant compounds means that new dopant compounds can be produced which may not be known as compounds suitable for catalysis. Generating a plurality of dopant compounds means that the compounds can be ranked and the most suitable compounds selected. A compound-property prediction machine learning model is used to predict properties of each dopant compound in the plurality of generated dopant compounds. Using a property prediction machine learning model for predicting properties means that properties can be accurately predicted. The plurality of generated dopant compounds are ranked based on the predicted properties and one or more dopant compounds are selected for catalysis based on the ranking. This means that dopant compounds with properties which make them suitable for catalysis can be selected.

Disclosed herein is an apparatus comprising: a processor, a memory storing instructions that, when executed by the processor, perform any of the methods described above.

Also disclosed is a computer storage medium having computer-executable instructions that, when executed by a computing system, direct the computing system to perform any of the methods described above.

### DESCRIPTION OF THE DRAWINGS

The present description will be better understood from the following detailed description read in light of the accompanying drawings, wherein:

FIG. 1 illustrates a generative model and a predictive machine learning model for determining a dopant package for catalysis;

FIG. 2 is a schematic diagram showing an example method of determining an output dopant package;

FIG. 3a shows example input dopant packages and interpolated dopant packages in an entire input space;

FIG. 3b shows example input dopant packages and interpolated dopant packages in a specific corner of the input space;

FIG. 4 illustrates the generation of a candidate dopant compound;

FIG. 5 is a schematic diagram showing the extraction of dopant information from one or more documents;

FIG. 6 shows an example user interface;

FIG. 7 is a flowchart of a computer-implemented method for determining a dopant package for catalysis;

FIG. 8 is a flowchart of a computer-implemented method for selecting one or more dopant compounds for catalysis; and

FIG. 9 illustrates an example computing device in which methods described herein are implemented.

Common reference numerals are used throughout the figures, where appropriate, to indicate similar features.

### DETAILED DESCRIPTION

The following description is presented in connection with the appended drawings and is intended as a description of the present examples to enable a person skilled in the art to make and use the invention. The description is not intended to represent the only forms in which the present examples are constructed or utilized. The present invention is not limited to the embodiments described herein and various modifications to the disclosed embodiments will be apparent to those skilled in the art.

As described above, the dopant package used in catalysis determines how effective catalysis is because dopants modify the performance of the catalyst. It is therefore desirable to find an effective dopant package. The present invention relates to catalysis of any chemical reaction which is catalyzed using a dopant package. An example of such a reaction is the production of ethylene oxide. As used herein, the term “dopant package” refers to amounts and types of one or more dopants and amounts and types of the main catalyst metal or metals. This may also be referred to as a catalyst formulation.

Dopants are additives in a catalyst formulation that modify the performance of the catalyst. Dopants are typically alkali metals, transition metals, halogens or compounds. Dopant packages are combinations of one or more dopants used in catalysis and one or more main catalyst metals. A dopant package includes a plurality of dopants each present in a dopant amount (that is, there is a ratio of dopants in the dopant package) as well as one or more catalyst metals each present in a certain amount. A dopant package has associated performance values which determine its suitability and/or effectiveness in catalysis.

Finding new effective dopant packages via known methods is a time-consuming process which may take decades. The present invention provides an automated method for determining a dopant package for catalysis which is faster and more efficient.

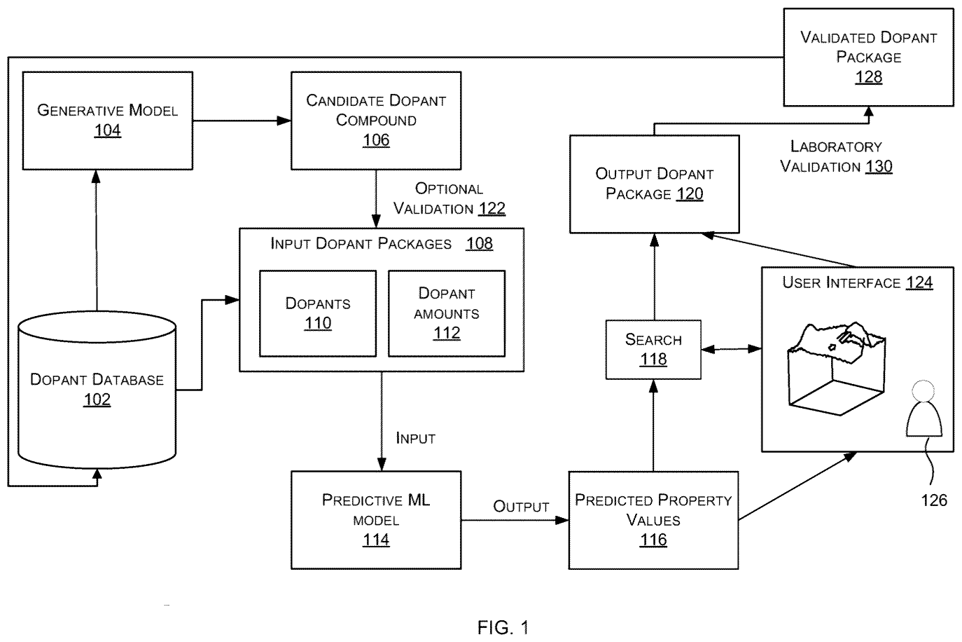

FIG. 1 shows a generative model 104 and a predictive machine learning (ML) model 114 for determining a dopant package for catalysis. The generative model 104 and the predictive ML model are computer implemented and are deployed on the same computing device or at different computing devices which are in communication with one another over a wired or wireless communications link. The generative model is obtained using a dopant database 102 which is a database storing information about dopants for catalysis including dopant compounds, descriptions of dopant compounds, performance values of dopant compounds, known dopant packages and their formulations and other information such as experiment conditions and carrier information.

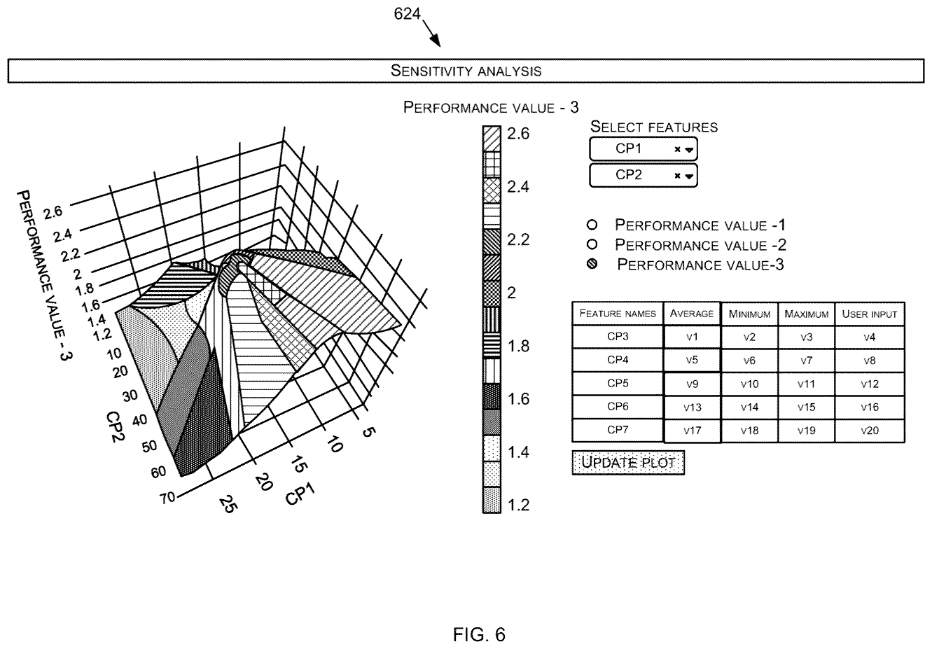

In various examples, a user 126 such as a scientist or engineer is able, via a user interface 124, to control the generative model 104 and the predictive ML model 114 in order to generate output dopant packages 120. More detail about the user interface 124 is explained with reference to FIG. 6. The user interface 124 is computer implemented and is functionality that has access to data including predicted performance values 116, input dopant packages 108, data from dopant database 102 and data relating to search 118. In various use scenarios, data are displayed to the user via the user interface (UI) 124 and the user edits the displayed data via user input. The UI 124 allows the user to inspect output data from predictive ML model 114 and/or generative model 104 in an interactive manner. The UI allows the user to explore patterns or trends in the data by selecting data to be displayed and providing input as to how the data is displayed. When search 118 is performed, as explained in more detail below, the UI may be used to provide details to the user about how the search is performed and to visualize the search. Additionally or alternatively, input provided by the user via the UI is used to perform the search; for example, the user provides search parameters or selects an output dopant package potentially from a plurality of offered candidate dopant packages.

The apparatus of FIG. 1 is usable to compute output dopant packages 120 using a process that includes two main parts. The first part involves generating a candidate dopant compound which is not included in dopant database 102 of known dopant compounds. The second part involves determining an output dopant package which includes the candidate dopant compound i.e. a combination of dopants, including the candidate dopant compound, which will be formulated and used in catalysis.

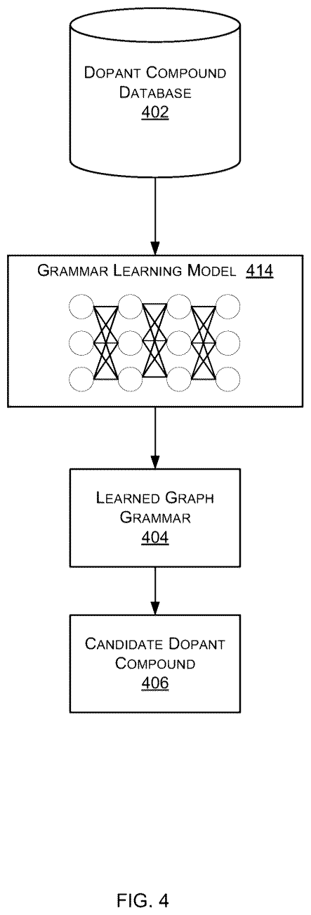

For the first part, generative model 104 is used to generate a candidate dopant compound 106. The generative model 104 produces new candidate compounds. The generative model in some examples is a learned graph grammar, which is described in more detail below with reference to FIG. 4. The graph grammar is learned from the known dopant compounds in database 102 with performance values associated with effective catalysis and relates to rules for compound construction. Database 102 includes information relating to dopant compounds including chemical and physical properties of molecules and chemical species and performance values of the dopant compounds. The dopant compounds which are used to learn the graph grammar have performance values which make the dopant compound suitable for catalysis and therefore the graph grammar learns how to construct compounds which are suitable for catalysis. In some examples, dopant compounds used to learn the graph grammar are selected from the dopant database 102 based on their performance values. Thus dopant database 102 stores, for each of a plurality of dopant compounds, a description of the dopant compound and a description of performance values of the dopant compound which make the dopant compound suitable for catalysis.

Once generated by the generative model, the candidate dopant compound 106 may be validated 122. Validation 122 in some examples comprises providing the candidate dopant compound to a large language model such as generative pretrained transformer GPT 4 (trade mark) or any other large language model (LLM). A non-exhaustive list of large language models which may be used is LLAMA, GEMINI, BLOOM, Mistral Large. A large language model is a machine learning model with around one billion parameters or more which is capable of generating language output. Validation is carried out automatically in some cases by generating a prompt using a prompt template. The prompt comprises an identifier of the candidate dopant compound and a request for one or more of: an indication of the suitability of the candidate dopant component for catalysis, information about availability of the candidate dopant compound, information about chemical properties of the candidate dopant compound. The LLM provides an indication of the suitability of the candidate dopant compound 106 for catalysis for example by providing information on the chemical properties of the candidate dopant compound, the availability of the candidate dopant compound or how to produce the candidate dopant compound. A response from the LLM is received and an automated process uses rules and the response to classify the candidate dopant compound as validated or not validated. In other examples the candidate dopant compound is validated by performing laboratory tests. In some cases the laboratory tests are automated. Where the candidate dopant compound fails validation generative model 104 is used to generate another candidate dopant compound.

Once the candidate dopant compound 106 has been generated and optionally successfully validated, an output dopant package 120 is determined in a second main part of the method. An output dopant package is a determined combination (formulation) of dopants and catalyst metals, where each dopant has a dopant amount. The output dopant package 120 is a dopant package suitable for catalysis and it is determined by performing a search 118 to find a dopant package with improved performance. The search is performed over a plurality of input dopant packages, the performance values of which are predicted using a predictive machine learning (ML) model 114.

Predictive ML model 114 takes as input an input dopant package. The model outputs predicted values of one or more performance values of the input dopant package. For example, an input dopant package contains X_a amount of dopant A, X_b amount of dopant B, X_c amount of dopant C, and X_m amount of a main catalyst metal M which is expressed as (X_a, X_b, X_c, X_m). The output of the model is performance values. For example, the predictive ML model outputs that the dopant package has a value D of performance value 1 and a value E of performance value 2. The predictive ML model is for example a random forest model, a neural network model, a model comprising boosted decision trees, a Catboost model, XgBoost model, Linear model, support vector machine (SVM), sparse Gaussian process regression, kernel ridge regression or other machine learning model.

The predictive ML model has been trained using a labeled training dataset which includes known dopant packages and their performance values i.e. known dopant packages and their associated performance values (inputs and outputs respectively of the predictive ML model 114). The model is trained using supervised learning based on the training set. If the predictive ML model is a neural network, suitable training methods include backpropagation to update weights and biases in the neural network model. If the predictive ML model is a random forest model the model is trained by, for each labeled training data item (dopant package and known performance values), passing the training data item from the root node of each tree in the forest to a leaf node of the tree by carrying out a test at each split node encountered on the route. The tests at the split node are learnt by selecting values of variables used in the tests and observing performance of the tests on a measure such as increased information gain. The training data item is stored at the leaf node it reaches. The process is repeated for each training data item and a concise representation of the training data items stored at each leaf node may be constructed, such as a variance and mean. During training the model parameters of the predictive ML model are adjusted.

After training, the predictive ML model 114 is used to generate predicted performance values for unseen dopant packages (i.e. dopant packages which were not part of the training dataset).

A plurality of input dopant packages 108 are obtained by selecting dopants at random from the dopant database 102 or using rules or other criteria to automatically select dopants from the dopant database 102. At least one of the input dopant packages includes the new candidate dopant compound 106. Each input dopant package includes dopants 110, and dopant amounts 112, wherein each dopant amount is an amount of the dopant in the package or a ratio of the dopant to other dopants in the package. In various examples, some of the input dopant packages contain the candidate dopant 106 as well as dopants from the dopant database 102. Dopant amounts are expressed for example as percentages by weight, or as percentages by surface area, or by molar quantities. Input dopant packages also include amounts of one or more main catalyst metals, where the amounts are expressed for example as percentages by weight.

The predictive machine learning (ML) model 114 produces the predicted values of performance measures 116 for catalysts doped using each of the input dopant packages. The predicted performance measures are for example catalyst selectivity or catalyst activity. The predictive machine learning model may be a random forest model, a neural network model, a model comprising boosted decision trees, a Catboost model, XgBoost model, Linear model, support vector machine (SVM) model, sparse Gaussian process regression, kernel ridge regression or other machine learning model. Predictive machine learning model 114 may be trained using supervised learning and a training dataset comprised of known dopant packages and their associated performance values.

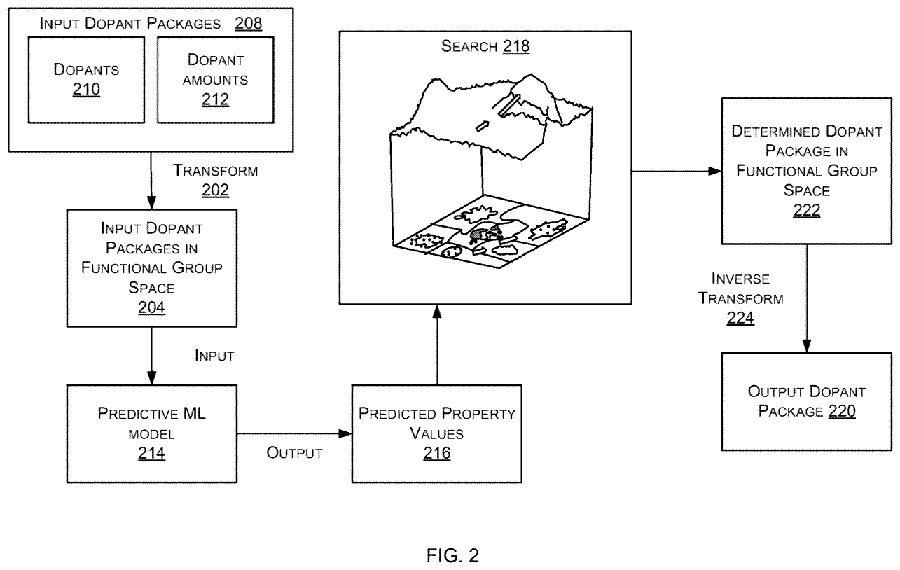

The predicted performance values are used to search 118 for an output dopant package 120. The search finds a dopant package with improved performance values. The search may involve identifying a trend in one or more of the performance values. In one example, performance value 1 is a desirable performance value, and an increasing trend is identified in performance value 1. The search result could be the dopant package with the maximum value in performance value 1 from the plurality of input dopant packages 108. Alternatively, the search result could be the result of interpolating the trend in performance value 1 in order to output a dopant package 120 which was not explicitly input into the predictive machine learning model 114. In another example, the search result could be obtained based on a negative trend in another performance value, performance value 2. In further examples, trends in multiple performance values are taken into account in order to determine the search result. In these examples as with the first example, the output dopant package may be a dopant package from the plurality of input dopant packages 108 or it could be the result of interpolation i.e. a dopant package which is not in the plurality of input dopant packages 108. Search 118 is described in more detail with reference to FIG. 2.

FIG. 2 is a schematic diagram showing an example method of determining an output dopant package 220 (which is an example of output dopant package 120). A plurality of input dopant packages 208 includes dopants 210 and dopant amounts 212 (these correspond to input dopant packages 108, dopants 110 and dopant amounts 112 in FIG. 1). In the example shown in FIG. 2, the input dopant packages 208 are transformed (202) into a functional group space. The transformation results in input dopant packages in functional group space 204.

Functional group space is a space with variables (dimensions) which correspond to functional groups of dopant compounds. Functional groups are constituents of a molecule which cause the molecule's chemical properties. An example of a functional group is an ion although there are many other types of functional group. In general the same functional groups undergo similar chemical reactions regardless of other parts of the molecule. Functional group space has reduced dimensionality in comparison to dopant space, in which each variable (dimension) corresponds to a dopant. By transforming into functional group space each dopant package may be represented as a combination of functional groups. Reducing dimensionality from dopant space to functional group space saves computational resources including storage and processing resources. For example the number of inputs into the predictive machine learning model 214, 114 is reduced and therefore fewer resources are used to predict performance values 216 for each input dopant package. In some examples, the variables of functional group space are determined by identifying functional groups in dopant compounds. For example, each dopant compound may be compared to a list of known functional groups in order to identify the functional groups present in the dopant compound.

Additionally, the dimensionality of functional group space can be further reduced using principal component analysis (PCA) or partial least squares (PLS). Both PCA and PLS reduce dimensionality of the functional group space by looking for linear combinations of the functional groups (i.e. variables in functional group space) which can be used to summarize the input data. Compared to PCA, PLS in addition takes into account the relationship between input and target variables. The variables resulting from PLS are called latent variables and the further reduced space is called latent variable space. Further reducing the dimensionality of functional group space saves more computational resources such as storage and processing resources.

The number of variables in functional group space can be determined based on percentage of cumulative explained variance of chemical properties from a known dataset. Known dopant packages and corresponding performance values may for example be part or all of the training dataset used to train predictive machine learning (ML) model 114, 214. In an example, the input dopant packages 208 are defined in terms of seven dopant compounds CP1-CP7. Each input dopant package contains different dopant amounts of dopants CP1-CP7. For example the input dopant package could be expressed as a seven-dimensional vector in dopant space. Transforming to functional group space to latent variable space with 3 variables, involving a dimensionality reduction from 7 to 3, accounts for 80% of the variance in chemical properties.

In the method shown in FIG. 2, the plurality of input dopant packages 208 are transformed 202 into functional group space. The transform 202 operation is automated and is carried out using an arithmetic operation such as addition or another form of aggregation. In other examples, input dopant packages are defined in functional group space.

Predictive ML model 214 predicts performance values 216 based on the input dopant packages. A search 218 is performed based on the predicted performance values in order to determine a dopant package. The aim of the search in some examples is to find a dopant package with one or more predicted performance values with values over a threshold or to find the dopant package with the maximum predicted value of one or more performance values. In various examples, the search is based on a trend in predicted performance values. This includes determining input dopant packages (in dopant space or in functional group space) based on a trend in performance values. For example, if a desirable performance value increases with one variable, then input dopant packages with higher values of that variable may be selected.

FIG. 2 is a schematic diagram to aid in explaining how the search 218 may be performed in functional group space. In FIG. 6, the value of a predicted performance values is plotted in the vertical axis, and functional group variables are represented on the horizontal axes. The plot represents variation in the performance values based on functional group variables. The predicted performance values are predicted by the predictive ML model 214. The search may involve finding the position in functional group space of the point with the maximum value of the performance value. The search is for example an interpolative search. Interpolation involves determining the performance value at a point in functional group space (which was not part of the plurality of input dopant packages). The performance value is found based on the performance value from points which were part of the plurality of input dopant packages.

Example search 218 includes two functional group variables and one performance value variable, however this is an example only and many other numbers of functional group variables and numbers of performance value variables are included in other scenarios. For example, there could be three functional group variables and three performance value variables.

In the example shown in FIG. 2 the search is performed in functional group space and the output of the search is a determined dopant package in functional group space 222. The determined dopant package in functional group space 222 is inverse transformed 224 back into dopant space in order to arrive at the output dopant package 220 which corresponds to output dopant package 120 in FIG. 1. The inverse transform 224 is automated and is a reverse of the operation 202. For example, where the transform 202 is performed using principal component analysis, the transform is performed via multiplication by the matrix W (the matrix of weights whose columns are eigenvectors). The inverse transform 224 is performed via a multiplication by W⁻¹ which is the inverse of W. Additionally or alternatively, the transform is performed by identifying functional groups from a list of functional groups which are present in each dopant compound. In these scenarios, the inverse transform is performed by combining functional groups resulting in a dopant compound. In other examples not shown in FIG. 2, the search is performed in dopant space.

In some examples, the predictive ML model predicts performance values based only on the input dopant package. In these examples, test conditions for catalysis may be predetermined. In other examples, the predictive ML model 214 is configured to take test conditions for catalysis as input. Examples of test conditions are flow rate, pressure and temperature. Any other suitable test condition may be included. The performance values are predicted based on a combination of dopant package and test conditions. The search in these scenarios does not necessarily take into consideration the test conditions. Performance metrics are compared or normalized to allow comparison between different test conditions in a consistent way.

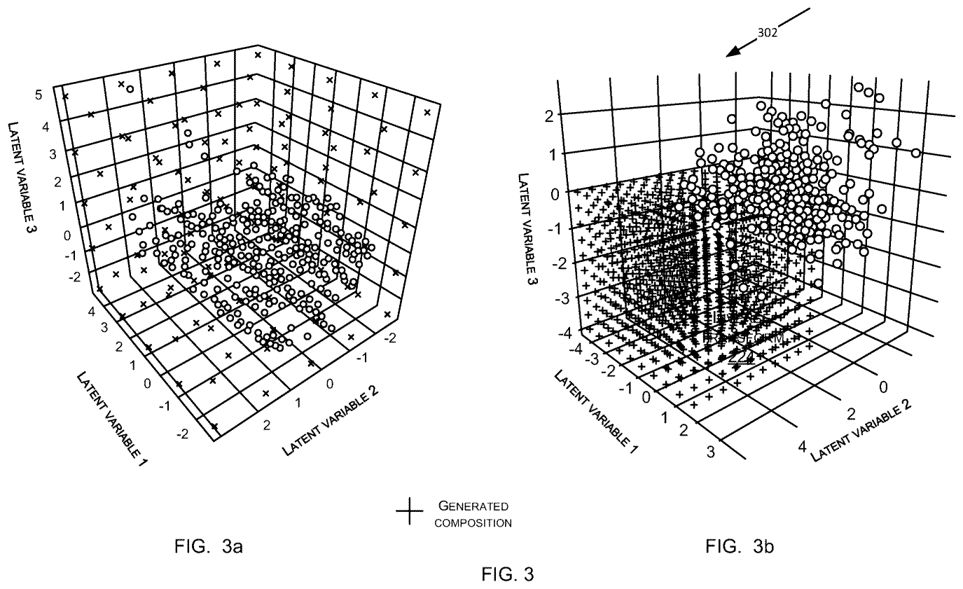

In further examples, the search is performed based on interpolating new dopant packages in latent variable space as shown in FIG. 3. As explained above, latent variable space results from reducing the dimensionality of functional group space. FIG. 3 includes two plots FIG. 3a and FIG. 3b showing example dopant packages in latent variable space. In these examples, latent variable space has three dimensions (latent variable 1, latent variable 2 and latent variable 3) and each dimension represents a linear combination of all functional groups. The plots in FIG. 3a and FIG. 3b include latent variable coordinates of known dopant packages shown as circular dots. Each dot has one or more corresponding performance values (not shown). These known dopant packages in some examples correspond to the training data for training predictive ML model 214 or 114. New interpolated dopant packages in latent variable space are plotted as black crosses.

In the example in FIG. 3a, the latent variable space is roughly spanned by the known dopant packages. New dopant packages (black crosses) are interpolated in the entire latent variable space. In FIG. 3b, the new dopant packages (black crosses) are interpolated in a region in latent variable space which extends beyond the space spanned by the known dopant packages. This is based on a trend in one or more performance values represented by arrow 302. The values of performance value 2 increase towards the region spanned by black crosses. In this and further examples, the input dopant packages in latent variable space are determined based on a trend in at least one performance value. This makes the method more efficient because the predictive ML model takes fewer inputs and predicts fewer values thus saving time and computational power. Because the inputs are selected based on a trend in a performance value, the inputs represent dopant packages which are likely to be effective in catalysis.

FIG. 4 illustrates the generation of a candidate dopant compound such as candidate dopant compound 106, 206. The candidate dopant compound is generated using generative model 104 as explained with reference to FIG. 1. In the example shown in FIG. 4 the generative model is a learned graph grammar 404. The learned graph grammar 404 includes production rules for generating dopant molecules. The dopant molecules are represented as graphs, where atoms correspond to nodes on the graph and chemical bonds correspond to edges of the graph. The graph grammar includes production rules for constructing a molecule represented as a graph. The graph grammar is learned using a training database of known dopant compounds 402 which is an example of dopant database 102. The graph grammar can be learned using a dopant database 402 which may contain for example tens or hundreds of known dopant compounds. The number of known dopants is limited because of the time it has taken to find suitable dopants using traditional discovery techniques (which can be decades). Therefore using a graph grammar 404 as a generative model 104 allows a candidate dopant compound 406, 106 to be generated using the limited training dataset comprising known dopant compounds in dopant database 402, 102.

The graph grammar in FIG. 4 is learned using a grammar learning model 414 which is shown in FIG. 4 as a neural network. Other suitable grammar learning models such as a Molecular Hypergraph Grammar (MHG) method are used in other examples. Grammar learning model 414 learns graph grammar 404 using dopant compounds selected from dopant database 402.

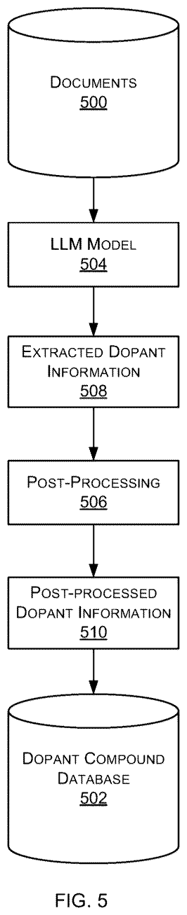

FIG. 5 is a schematic diagram showing the extraction of dopant information from one or more documents. Documents 500 in FIG. 5 include documents such as patent documents, scientific papers and scientific reports relating to dopants for catalysis. Documents 500 are input into an LLM (large language model) 504 in order to extract dopant information from the documents. The LLM is for example ChatGPT, BLOOM, LlaMA, Mistral Large or another large language model which has been trained on a very large corpus of training data such as the internet or a corpus of documents about dopants. The documents may be provided with a user-generated prompt in order to extract relevant information from the documents. Examples of prompts include “Could you locate ‘DETAILED DESCRIPTION’ and give me a summary of the text after it?”, “Could you locate ‘BACKGROUND’ and give me a summary of the text after it?” and “Could you please give me the paragraph under FIELD OF INVENTION?”. Sometimes, dopant information is extracted from a data table in documents 500 using the LLM.

LLM model 504 extracts dopant information 508 after which post-processing 506 may be performed. Post-processing may include a similarity search against items already in the dopant database 502 to reduce redundancy in the dopant database 502, text vectorization or filtering. Dopants extracted from documents 500 which are already in dopant database 502 are not added. In FIG. 5, post-processed dopant information 510 is included in dopant database 502 which corresponds to dopant database 102 in FIG. 1 and dopant database 402 in FIG. 4.

FIG. 6 shows an example user interface (UI) 624 which is one example of user interface 124 described above with reference to FIG. 1. As described with reference to FIG. 1, the user interface 124 is computer implemented and is functionality that has access to data including predicted performance values 116, input dopant packages 108, data from dopant database 102 and data relating to search 118. In various use scenarios, data are displayed to the user via the user interface (UI) 124 and the user edits the displayed data via user input. The UI 124 allows the user to inspect output data from predictive ML model 114 and/or generative model 104 in an interactive manner. The UI allows the user to explore patterns or trends in the data by selecting data to be displayed and providing input as to how the data is displayed. When search 118 is performed, as explained in more detail below, the UI may be used to provide details to the user about how the search is performed and to visualize the search. Additionally or alternatively, input provided by the user via the UI is used to perform the search.

The example UI shown in FIG. 6 allows the user to provide input and also displays predicted values of the one or more performance values from the predictive ML model 114, 214. In FIG. 6, two variables in dopant space, compound 1 (CP1) and compound 2 (CP2), are plotted on the x and y axes and a performance value (performance value-3) is plotted on the z axis and using a color scale. The plotted values of performance value-3 are predicted values from predictive machine learning model 114, 214. In other examples the properties are plotted in functional group space. The UI allows the user 126 to visualize predicted performance values. Features to plot are selected by the user and in this example CP1 and CP2 are selected for display. Performance values to plot are also selected by the user; in this example performance value-3 is selected from options of performance value-1, performance value-2, performance value-3. In the example of FIG. 6 there are 7 features. Two features CP1, CP2 are selected in dropdown boxes. The rest of the features are shown in the table with their average, minimum and maximum values which correspond to dopant amounts. In the list column of the table a user is able to input their own values for each feature directly into the table. The average, minimum, maximum, and user input values for CP3-CP7 are shown as v1-v20 in FIG. 6. The user may update the plot once they have selected the desired input using the “Update plot” button shown in FIG. 6.

In the example shown in FIG. 6, the UI 624 displays information about how performance values change in response to variables corresponding to dopant compounds. The UI displays predicted performance values as well as input dopant package data in order for the user to see and explore the relationship between the dopant package and the performance values e.g. as a graph. In FIG. 6, dopant package data is displayed in dopant space but the data may also be displayed in functional group space. Although not shown in FIG. 6, the UI may display on the same plot the training dataset used to train predictive ML model 114 i.e. known combinations of dopant package and associated performance values. This allows the user to compare the performance values of known dopant packages against the performance values of new dopant packages.

In further use scenarios, user 126 uses various examples of the UI 124 to visualize, inspect and analyze data including: data relating to input dopant packages, predicted performance values from the predictive ML model, data stored in the dopant database 102, data relating to the candidate dopant compound 106, training data for the predictive ML model 114 and data relating to search 118. In various examples the user selects which data are displayed and how the data are displayed including type of plot, colors, scales and other display variables. The user may interact with the display for example by resizing the display.

In other example user interfaces (which are further examples of user interface 124), a UI allows the user to provide various forms of input which can be used to determine the output dopant package. In some examples, the user provides input, via the UI, which is used to determine the plurality of input dopant packages 108, 208, 204 to be input into the predictive ML model 114, 214. The user input may relate to an area in functional group space or dopant space in which the input dopant packages lie. The area in functional group space may be selected with mouse clicks or by entering ranges into the UI using the computer keyboard. The number of input dopant packages, the performance value on which to base the search, or parameters relating to the search such as whether the search is interpolative may also be input by the user in order to determine the input dopant packages. In some scenarios the user input determines the output dopant package 120 based on selection by the user for example by the user selecting a point on a graph corresponding to a dopant package by clicking with a mouse.

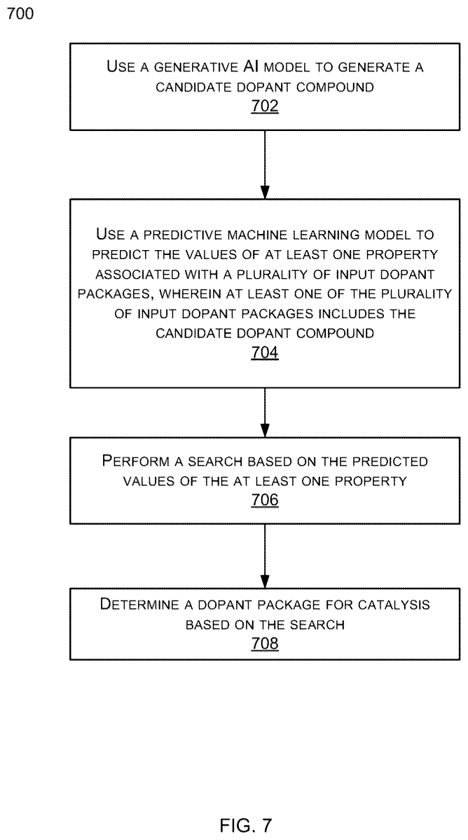

FIG. 7 is a flowchart of a computer-implemented method 700 for determining a dopant package for catalysis. The method of FIG. 7 is performed by a computing entity in communication with the generative AI model and the predictive ML model. At block 702 a generative model is used to generate a candidate dopant compound such as dopant compound 106 or 406. The candidate dopant compound is generated for example by the method described with reference to FIG. 4 above. At block 704, a predictive ML model is used to predict the values of one or more performance values associated with a plurality of input dopant packages, wherein at least one of the plurality of input dopant packages includes the candidate dopant compound. The predictive ML model is for example predictive ML model 114, 214 and may be a neural network model, a random forest model, a model comprising boosted decision trees, a Catboost model, XgBoost model, Linear model, support vector machine (SVM), sparse Gaussian process regression, kernel ridge regression or other machine learning model. At block 706 a search is performed based on the predicted values of the one or more performance values and at block 708 a dopant package is determined based on the search performed at block 706. The steps performed at blocks 704, 706 and 708 are described in more detail with respect to FIG. 2 above.

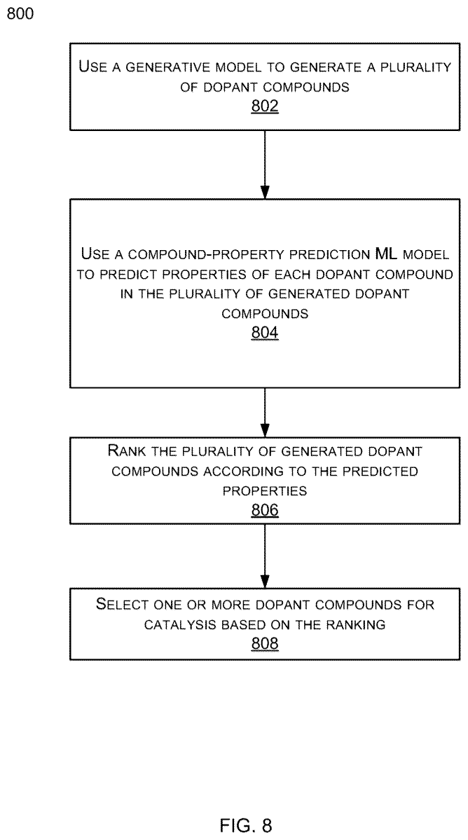

FIG. 8 is a flowchart of a computer-implemented method 800 for selecting one or more dopant compounds for catalysis. The method includes using a generative model to generate a plurality of dopant compounds, 802. The generative model may be generative model 104, or 404 and the dopant compounds may be generated using the method shown in FIG. 4. In various examples, the generative model is a learned graph grammar such as learned graph grammar 404 which is described in more detail with reference to FIG. 4 above. At block 802, a plurality of dopant compounds are generated by the generative model. Multiple dopant compounds are generated so that they can be ranked and the most suitable dopant compound or compounds selected.

The plurality of generated dopant compounds are ranked based on predicted properties of each compound as shown at block 806. The predicted properties are predicted based on a compound-property prediction machine learning (ML) model as shown at 804. The properties relate to suitability for use as a dopant for catalysis. Examples of predicted properties include: electrical conductivity, pKa, melting point, glass transition temperature, boiling point, dipole moment, quadruple moment, vapor pressure, heat of formation, heat of combustion, solubility, lipophilicity, surface adsorption energy, dielectric constant, diffusion constant, chemical hardness, polarizability, heat of vaporization, heat of melting, viscosity, viscoelasticity, refractive index, magnetic susceptibility, heat capacity, HOMO-LUMO (highest occupied molecular orbital-least unoccupied molecular orbital) gap, electrophilicity, partition coefficient, protein-ligand binding affinity, Fluorescence Lifetime, oxidation potential, molecular conformational rigidity, isoelectric point, ionic radius, ionic charge, molecular shape, electronegativity, standard ionization potential, density, nuclear magnetic resonance (NMR) spectral features, infrared spectral features, ultraviolet-visible spectral features, atomic weight, molecular weight, or any other suitable property.

The compound-property prediction ML model in examples is a graph neural network (GNN) or a gradient boosting machine or any other suitable machine learning model. The property prediction ML model is trained using training data comprising known dopant compounds and their respective properties. The training data may be included in dopant database 102, 402 and the property prediction ML model is trained using supervised learning. During training, the model parameters are adjusted such that the machine learning model accurately predicts one or more outcome properties based on an input dopant compound. If the compound-property prediction ML model is a neural network, suitable training methods include backpropagation to update weights and biases in the neural network model. The compound-property prediction ML model takes as input a dopant compound and outputs predicted compound properties.

Once the properties of each generated dopant compound have been predicted, the dopant compounds are ranked according to the predicted properties. The compounds are ranked based on one or more properties. In some examples, the dopant compounds are ranked according to one property (which may be considered the most important property) and the dopant compound which is ranked first has the highest, or lowest, predicted value of that property. In other examples, the dopant compounds are ranked according to multiple properties, which may be weighted such that higher weights correspond to more important properties. In various scenarios, the ranking is performed using an objective function which may be created using one or more properties. Sometimes a single property is used as an objective function and sometimes an objective function includes more than one property. The objective function may include some or all of the predicted properties. For example, the higher the value of the objective function, the higher the ranking. Alternatively, the lower the value of the objective function, the higher the ranking.

In some examples, dopant compounds are included or excluded from the ranking based on setting thresholds for one or more properties. Thresholds represent the range of property values determined to be suitable for catalysis. For example, some properties have a corresponding upper threshold and lower threshold. Property values below the upper threshold and above the lower threshold are suitable for catalysis. In further examples, a property has only an upper threshold and only property values below the upper threshold are suitable. In another example, a property has only a lower threshold and only property values above the upper threshold are suitable. Some properties may have multiple suitable ranges. In these examples, dopant compounds with one or more predicted property values lying outside the corresponding suitable range(s) are excluded from the ranking. Dopant compounds with predicted property values lying inside the corresponding suitable range(s) are included in the ranking.

In various scenarios, dopant compounds with one or more predicted property values lying outside the determined suitable range(s) are excluded from the ranking. Then, ranking is performed on the remaining dopant compounds based on an objective function comprising either one property or more than one property as described above. In other scenarios, the ranking is performed without including or excluding dopant compounds based on threshold values.

One or more dopant compounds are selected based on the ranking at block 808. For example, the top 1, 2, or n compounds are selected from a ranked list of dopant compounds. In other examples, the dopant compounds may be selected based on threshold values of one or more predicted properties. The method 800 therefore provides new suitable dopant compounds for catalysis.

In some scenarios, the selected dopant compounds which are the result of method 800 are included in one or more input dopant packages such as input dopant packages 108 described above with reference to claim 1. Predictive ML model 114 predicts property values which are associated with an input dopant package. Search such as search 118 may then be used to find the most suitable dopant package. Alternatively or additionally, method 700 may be adapted to include generating a plurality of dopant compounds, 802, predicting properties of each dopant compound in the plurality of generated dopant compounds, 804, ranking the plurality of generated dopant compounds according to the predicted properties, 806, and selecting one or more dopant compounds for catalysis based on the ranking, 808. By leveraging the use of a compound-property prediction ML model (which determines properties of individual dopant compounds) and predictive ML model 114 (which determines properties of a dopant package), a more suitable dopant package can be determined. This is because dopant compounds with more suitable individual properties are more likely to result in improved dopant packages.

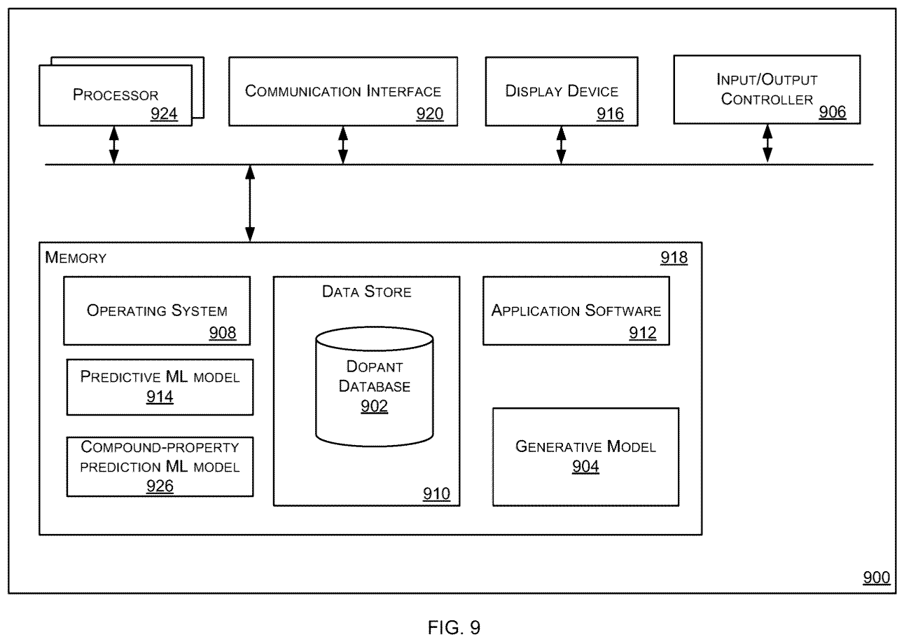

FIG. 9 illustrates an example computing device 900 in which methods described herein are implemented. Computing-based device 900 comprises one or more processors 924 which are microprocessors, controllers, or any other suitable type of processors for processing computer executable instructions to control the operation of the device in order to determine a dopant package and/or to select one or more dopant compounds for catalysis. In some examples, for example where a system on a chip architecture is used, the processors 924 include one or more fixed function blocks (also referred to as accelerators) which implement a part of the methods disclosed herein in hardware (rather than software or firmware). Platform software comprising an operating system 908 or any other suitable platform software is provided at the computing-based device to enable application software 912 to be executed on the device. A data store 910 holds for example dopant database 902, additionally or alternatively predicted performance values, a plurality of generated dopant compounds, predicted properties of the generated dopant compounds and rankings of the generated dopant compounds, documents or any type of data suitable for determining a dopant package for catalysis and/or selecting one or more dopant compounds for catalysis. Predictive ML Model 914 which in some examples is predictive ML model 114, 214 and generative model 904 which in some examples is generative model 104, 404 are also stored in memory 918 to be used to determine a dopant package for catalysis. In some examples, a compound-property prediction ML model 926 which predicts properties of a dopant compound is stored in memory 918.

The computer executable instructions are provided using any computer-readable media that are accessible by computing based device 900. Computer readable media include, for example, computer storage media such as memory 918 and communications media. Computer storage media, such as memory 918, include volatile and non-volatile, removable and non-removable media implemented in any method or technology for storage of information such as computer readable instructions, data structures, program modules or the like. Computer storage media includes, but is not limited to, random access memory (RAM), read only memory (ROM), erasable programmable read only memory (EPROM), electronic erasable programmable read only memory (EEPROM), flash memory or other memory technology, compact disc read only memory (CD-ROM), digital versatile disks (DVD) or other optical storage, magnetic cassettes, magnetic tape, magnetic disk storage or other magnetic storage devices, or any other non-transmission medium that is used to store information for access by a computing device. In contrast, communication media embody computer readable instructions, data structures, program modules, or the like in a modulated data signal, such as a carrier wave, or other transport mechanism. As defined herein, computer storage media does not include communication media. Therefore, a computer storage medium should not be interpreted to be a propagating signal per se. Although the computer storage media (memory 918) is shown within the computing-based device 900 it will be appreciated that the storage is, in some examples, distributed or located remotely and accessed via a network or other communication link (e.g. using communication interface 920). The computing-based device 900 also comprises an input/output controller 906 arranged to output display information to a display device 916 which may be separate from or integral to the computing-based device 900. The display information may provide a user interface and/or parameter values. The input/output controller 906 is also arranged to receive and process input from one or more devices, such as a user input device (e.g. a mouse, keyboard, camera, microphone or other sensor). In some examples the user input device detects input from a user which is used in a search to find a dopant package.

The term ‘computer’ or ‘computing-based device’ is used herein to refer to any device with processing capability such that it executes instructions. Those skilled in the art will realize that such processing capabilities are incorporated into many different devices and therefore the terms ‘computer’ and ‘computing-based device’ each include personal computers (PCs), servers, mobile telephones (including smart phones), tablet computers, set-top boxes, media players, games consoles, personal digital assistants, wearable computers, and many other devices.

The methods described herein are performed, in some examples, by software in machine readable form on a tangible storage medium e.g. in the form of a computer program comprising computer program code means adapted to perform all the operations of one or more of the methods described herein when the program is run on a computer and where the computer program may be embodied on a computer readable medium. The software is suitable for execution on a parallel processor or a serial processor such that the method operations may be carried out in any suitable order, or simultaneously.

Those skilled in the art will realize that storage devices utilized to store program instructions are optionally distributed across a network. For example, a remote computer is able to store an example of the process described as software. A local or terminal computer is able to access the remote computer and download a part or all of the software to run the program. Alternatively, the local computer may download pieces of the software as needed, or execute some software instructions at the local terminal and some at the remote computer (or computer network). Those skilled in the art will also realize that by utilizing conventional techniques known to those skilled in the art that all, or a portion of the software instructions may be carried out by a dedicated circuit, such as a digital signal processor (DSP), programmable logic array, or the like.

Any range or device value given herein may be extended or altered without losing the effect sought, as will be apparent to the skilled person.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items.

The operations of the methods described herein may be carried out in any suitable order, or simultaneously where appropriate. Additionally, individual blocks may be deleted from any of the methods without departing from the scope of the subject matter described herein. Aspects of any of the examples described above may be combined with aspects of any of the other examples described to form further examples without losing the effect sought.

The term ‘comprising’ is used herein to mean including the method blocks or elements identified, but that such blocks or elements do not comprise an exclusive list and a method or apparatus may contain additional blocks or elements.

It will be understood that the above description is given by way of example only and that various modifications may be made by those skilled in the art. The above specification, examples and data provide a complete description of the structure and use of exemplary embodiments. Although various embodiments have been described above with a certain degree of particularity, or with reference to one or more individual embodiments, those skilled in the art could make numerous alterations to the disclosed embodiments without departing from the scope of this specification.

We claim:

1. A computer implemented method for determining a dopant package for catalysis, wherein the dopant package comprises one or more dopants, each dopant having a dopant amount, the method comprising:
using a generative model to generate a candidate dopant compound;
using a predictive machine learning model to predict performance values associated with a plurality of input dopant packages, wherein at least one of the plurality of input dopant packages includes the candidate dopant compound; and
determining the dopant package for catalysis by performing a search based on the predicted performance values.

2. The method of claim 1, wherein the plurality of input dopant packages are transformed into a functional group space or wherein the plurality of input dopant packages are defined in the functional group space, wherein dimensions in the functional group space correspond to functional groups.

3. The method of claim 2, wherein the search is performed in functional group space.

4. The method of claim 1 wherein the search is an interpolative search.

5. The method of claim 1 wherein the search is based on a trend in the performance values.

6. The method of claim 1 wherein the plurality of input dopant packages are determined based on a trend in known performance values.

7. The method of claim 1, further comprising determining one or more test conditions for catalysis by providing a plurality of test conditions to the predictive machine learning model along with the input dopant packages, wherein the predictive machine learning model predicts the performance values based on the input test conditions.

8. The method of claim 1, wherein the predictive machine learning model is a random forest model, a neural network model or a model comprising boosted decision trees, and/or wherein the predictive machine learning model is trained in a supervised manner on a dataset of dopant packages and performance values associated with each dopant package.

9. The method of claim 1, wherein the performance values are related to chemical properties associated with catalysis comprising activity or selectivity.

10. The method of claim 1, further comprising displaying the predicted values of the performance values from the predictive machine learning model in a user interface UI.

11. The method of claim 10 wherein a user provides input to the UI and the user provided input is used to determine the dopant package.

12. The method of claim 1 wherein the generative model comprises a learned graph grammar for dopant compounds, wherein the learned graph grammar includes production rules for generating dopant compounds.

13. The method of claim 1 further comprising validating the candidate dopant compound by inputting the candidate dopant compound into a large language model.

14. The method of claim 1, further comprising:
obtaining the generative model using a dopant compound training dataset comprising a plurality of dopant compounds,
wherein at least one of the plurality of dopant compounds in the dopant compound training set is determined by using a large language model to extract dopant information from one or more documents.

15. A computer implemented method for selecting one or more dopant compounds for catalysis, the method comprising:
generating a plurality of dopant compounds using a generative model;
using a compound-property prediction machine learning ML model to predict one or more properties of each dopant compound in the plurality of generated dopant compounds;
ranking the plurality of generated dopant compounds according to the predicted one or more properties;
selecting one or more dopant compounds for catalysis based on the ranking.

16. An apparatus for determining a dopant package for catalysis, wherein the dopant package comprises one or more dopants, each dopant having a dopant amount, the apparatus comprising a processor and a memory, the memory storing instructions which when executed on the processor:
use a generative model to generate a candidate dopant compound;
use a predictive machine learning model to predict performance values associated with a plurality of input dopant packages, wherein at least one of the plurality of input dopant packages includes the candidate dopant compound; and
determine the dopant package for catalysis by performing a search based on the predicted performance values.

17. The apparatus of claim 15 wherein the plurality of input dopant packages (208) are transformed into a functional group space or wherein the plurality of input dopant packages are defined in the functional group space, wherein dimensions in the functional group space correspond to functional groups.

18. The apparatus of claim 15 wherein the search is performed in functional group space.

19. The apparatus of claim 15 wherein the search is an interpolative search

20. The apparatus claim 15 further comprising determining one or more test conditions for catalysis by providing a plurality of test conditions to the predictive machine learning model along with the input dopant packages, wherein the predictive machine learning model predicts the performance values based on the input test conditions
